(19) United States
(12) Patent Application Publication
(10) Pub. No.: US 2011/0087326 A1
(43) Pub. Date: Apr. 14, 2011

(54) METHOD & APPARATUS FOR ARTHROSCOPIC BICEPS TENODESIS

(76) Inventor: Lonnie E. PAULOS, Pensacola Beach, FL (US)

(21) Appl. No.: 12/969,566

(22) Filed: Dec. 15, 2010

Related U.S. Application Data
(63) Continuation-in-part of application No. 12/011,455, filed on Jan. 28, 2008, now abandoned.

(51) Int. Cl. A61F 2/08

(52) U.S. Cl. 623/13.14

(57) ABSTRACT

A method and apparatus for arthroscopic biceps tenodesis wherein the method involves arthroscopically determining that biceps tenodesis is desirable; arthroscopically separating the long head of the biceps tendon from bone attachment; creating and placing a wedge at the proximal end of the long head of the biceps tendon; and allowing the tendon to slide through the glenohumeral joint and the wedge structure to be wedged in the bicipital groove. In some embodiments, the apparatus includes a button clamp wedge having top and bottom portions having mating protrusions and recesses to lock the top and bottom portions together, such that staggered ribs on the top portion and bottom portion grip top and bottom surfaces of the long head of the tendon when the top and bottom portions of the button clamp wedge are locked together. Some embodiments of the button clamp wedge have hinged portions similar to a clamshell.

### CROSS-REFERENCE TO RELATED APPLICATIONS

This application is a Continuation-In-Part application of and claims the benefit of co-pending U.S. app. Ser. No. 12/011,455, filed on Jan. 28, 2008, entitled “Method & apparatus for arthroscopic biceps tenodesis,” the entire contents of which is incorporated herein by reference in its entirety.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

1. Field of the Invention

This invention relates to methods and apparatuses for performing an arthroscopic biceps tenodesis.

2. Description of the Prior Art

With the increased use of systematic arthroscopic shoulder examinations and arthroscopic procedures, pathology of the long head of the biceps tendon has become more frequently identified. Biceps tendon pathology is found in a wide variety of shoulder conditions, ranging from instability to rotator cuff disorders and primary biceps tendon pathology. Prior to arthroscopic viewing of the glenohumeral joint and the detailed examination that it affords, the long head of the biceps was rarely visualized surgically and biceps tendon pathology was not recognized until in later stages. Arthroscopy allows earlier recognition and treatment of biceps pathology. While the definitive role of the long head of the biceps tendon remains controversial, its importance as a contributor of shoulder dysfunction has been increasingly recognized.

Treatment options for the long head of the biceps pathology initially consists of observation or non-operative management during early stages or upon initial presentation. Known surgical options initially include debridement of frayed tendinous portions. Tenotomy treatment has usually been reserved for older, less active patients with chronic shoulder pain. In some cases, tenotomy has been used in connection with massive, irreparable rotator cuff tears. Tenodesis of the biceps tendon is commonly indicated for younger, more active patients because it affords a primary advantage of improved cosmesis.

Open techniques are well known and provide secure fixation for the transplanted biceps tendon. Such open techniques, however, have potential major disadvantages since morbidity may be incurred through necessary accessory incisions. More recently, some arthroscopic procedures have been proposed. The advantages of use of arthroscopic procedures over open techniques include decreased morbidity resulting from open incisions and more rapid rehabilitation. Disadvantages are the increased operative time required and, currently, a lack of knowledge of the long term success of the treatments.

It is well recognized that a known process identified as “keyhole” tenodesis, may be used and be performed either arthroscopically or open and is often used in the repair of rotator cuffs. In addition, it is sometimes used as an isolated procedure with what is called “interval lesions”. Furthermore, even isolated biceps pathology, on occasion, requires some form of tenodesis.

At the present time there is available, on the market, an interference screw that is sold for anchoring a tendon end to bone during keyhole biceps tenodesis. Unfortunately, the interference screw causes tendon damage and may amputate the tendon at the edge of the bone panel. The tendon then tends to degenerate. It is also becomes very difficult to even place sutures that will hold the tendon during healing. Consequently, it is necessary that during an operation, special suturing techniques be used to try and fix a degenerate tendon. The healing of a sutured tendon takes much longer and requires protection, with a resultant loss of motion and with a potential need for further surgery that will include follow-up scope evaluation and manipulation of the tendon.

### BRIEF SUMMARY OF THE INVENTION

Principal objects of the invention are to provide biceps tenodesis that is safe, effective and reproducible. Other objects are to provide a procedure that can be performed quickly and without the need for specialized equipment. At most, a very simple, easily used anchor button may be employed as the wedging structure placed at the proximal end of the long head of the biceps tendon. Accessory incisions are not required and operative time and costs are reduced in comparison with other possible procedures.

It is believed that the procedure of creating and using a wedging structure in biceps tenodesis can successfully improve pain control, while still resulting in minimal cosmetic deformity and/or loss of strength of the bicep.

Wedging tenodesis requires an initial arthroscopic assessment of the biceps tendon. If appropriate significant symptoms and pathology are found to be present, the wedging tenodesis may be performed. A grasping monofilament stitch is placed into the proximal aspect of the biceps tendon in known fashion, through a cannula that is typically placed in the rotator interval and immediately anterior to the AC joint. The suture is then retrieved through the cannula, i.e., an 8 mm cannula, placed through an accessory anterolateral portal. Thereafter, the long head of the biceps is released from its attachment on the superior labrum.

The released tendon is delivered into the anterolateral cannula and the anterolateral cannula is withdrawn, thus, pulling the tendon out the anterolateral portal. A clamp is placed across the tendon approximately three cm from its end to prevent it from prematurely retracting back into the shoulder.

A wedging structure is affixed to the remaining stump of tendon and in a preferred embodiment the wedging structure may be fashioned from the stump end of the tendon by forming the end as a knot. The knot may be formed and tied as an overhead knot in the stump end of the tendon. Alternatively, a knot may be formed with braided suture securing a rolled top of the stump end. The knot formed in the tendon needs to be large enough to catch on the end of the intertubercular groove, in order to provide an effective tenodesis. The glenohumeral joint is then visualized arthroscopically from the posterior portal and the biceps tendon is allowed to retract into the intertubercular groove. The knot in the tendon secures it at the proximal aspect of the groove and prevents continued tendon retraction. After satisfactory placement has been confirmed, all suture strands are arthroscopically cut. The wedging tenodesis is performed in advance of other shoulder pathology, such as repair of rotator cuff tears.

Another embodiment of wedging tenodesis involves use of a button clamp-type wedge in place of a formed knot, as previously described.

In this embodiment, the procedure is as previously described, except that rather than formation of a knot in the stub end of the tendon, a button clamp wedge is attached to the stub end of the tendon. With these embodiments, the tendon is secured by lodging the button clamp wedge in the intertubercular groove. The button clamp wedge comprises a clamshell type button having an elongate, generally football shaped configuration. Other types of button clamps are suitable and can include, but are not limited to those described herein.

The clamshell type button of the invention includes a top section and a bottom section connected at one end of the football shaped body by a hinge and with locking holding means at the opposite locking end of the body to secure the top and bottom sections together in a clamping configuration. A hole through which a traction suture is passed is formed in the hinge end of the bottom section. A rack having spaced teeth along its length is formed at the locking end of the bottom section. The rack pivots at the locking end of the bottom section to extend into and through an opening formed at the locking end of the top section. A shaped locking flange formed to extend into the opening at the locking end of the top section is sufficiently flexible to allow insertion of the rack into the opening and, because of the shape prevents withdrawal of the inserted rack. Once the rack is pushed into the opening the top and bottom sections are securely locked together. The long head end of the biceps tendon being repaired is positioned between the top and bottom sections and when the top and bottom portions are locked together, the clamshell type button is firmly secured to the tendon.

The outer exterior surfaces of the top and bottom portions are preferably made “rough” to create a roughened exterior surface to enhance wedging during use. In addition, staggered clamping ribs on the engaging surfaces of the top and bottom sections are pushed into the tendon to further insure secure attachment of the clamshell type button to the tendon and to prevent sliding of the button along and off the tendon.

It is also an object of one embodiment of the invention to provide a button clamp wedge for use in performing wedge tenodesis of a biceps tendon comprising a clamp body having a separable first and second portion, the first and second portions each having an exterior and an interior surface. a means for locking the first and second portions together, a gripping means extending from the interior surface of the first and second portion and each of the gripping means having an apex extending towards the other portion when the portions are locked together. In some embodiments, when the portions are locked together the apex of the gripping means on the first portion and the apex of the gripping means on the second portion alternate along a length of the portion bodies. In some embodiments, the exterior surface of the portions is roughened.

It is another object of one embodiment of the invention to provide a button clamp wedge wherein the means of locking the first and second portions together comprises a magnetized north and south pole section on each of the portions whereby placing oppositely magnetized sections adjacent to each other cause the sections to attract each other and lock the first and second portion together.

It is another object of one embodiment of the invention to provide a button clamp wedge wherein the means of locking the first and second portions together, comprises a protrusion extending away from the body of the first portion, a recess extending into the body of the second portion and the first portion protrusion having a shape to be received and frictionally held in the second portion recess whereby the first and second portions can be connected.

Additional objects and features of the invention will become apparent to persons skilled in the art to which the invention pertains from the following detailed description and claims.

### BRIEF DESCRIPTION OF THE SEVERAL VIEWS OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1, is a pictorial view of a long head of a biceps tendon arthoscopically end trimmed and pulled through a portal to be prepared for a wedge tenodesis;

FIG. 2, a pictorial view of a biceps tendon with a tied knot formed therein during wedge tenodesis;

FIG. 3, a similar view showing formation of a sutured knot formed in a biceps tendon during wedge tenodesis;

FIG. 4, a wedge tenodesis with a knot wedged in the bicipital groove;

FIG. 5, a pictorial view of a tenodesis having a knot formed in the end of a biceps tendon and showing the knot arranged to be wedged into a keyhole opening of the type commonly formed in the humeras in preparation for a “keyhole” tenodesis;

FIG. 6, a pictorial view showing how a knotted end of a biceps tendon is moved into a keyhole opening;

FIG. 7, a similar view showing the knotted end of a biceps tendon wedged into a keyhole opening;

FIG. 8, a view like that of FIG. 6, showing how the knotted end is positioned in a keyhole opening;

FIG. 9, a pictorial view showing securement of a clamshell type button clamp wedge to a biceps tendon;

FIG. 10, a view like that of FIG. 9, showing a clamshell type button clamp wedge clamped to a biceps tendon and wedged into a bicipital groove;

FIG. 11, a top plan view of the closed button clamp wedge of FIG. 10;

FIG. 12, a side elevation view of the button clamp wedge of FIG. 10, showing the wedge in a clamping, locked position;

FIG. 13, a longitudinal section taken on the line 13-13 of FIG. 11;

FIG. 14, a view like that of FIG. 12, but showing the button clamp wedge in a partially open condition and with a biceps tendon laid over the bottom portion of the button clamp wedge;

FIG. 15, an enlarged sectional view taken on the line 15-15 of FIG. 9;

FIG. 16, a perspective view of one embodiment of a two-piece button with the pieces turned open to show details;

FIG. 17, a perspective view of another embodiment of a two-piece button with the pieces turned open to show details;

FIG. 18A, a side view of one embodiment of a two piece button with the pieces open;

FIG. 18B, a bottom view of one embodiment of a top piece of a two piece button;

FIG. 19, a perspective view of another embodiment of a two-piece button wherein the means for locking is a magnetic section;

FIG. 20, a perspective view of one embodiment of a single portion of a two-piece button; and

FIG. 21 illustrates a perspective view of another embodiment of a two-piece button with treaded locking means.

### DETAILED DESCRIPTION OF THE INVENTION

Methods and apparatuses for arthroscopic biceps tenodesis will now be described in detail with reference to the accompanying drawings. It will be appreciated that, while the following description focuses on an assembly for use with a biceps, the systems and methods disclosed herein have wide applicability. For example, the methods and apparatus described herein may be readily employed with other muscle or tendon tissue. Notwithstanding the specific example embodiments set forth below, all such variations and modifications that would be envisioned by one of ordinary skill in the art are intended to fall within the scope of this disclosure.

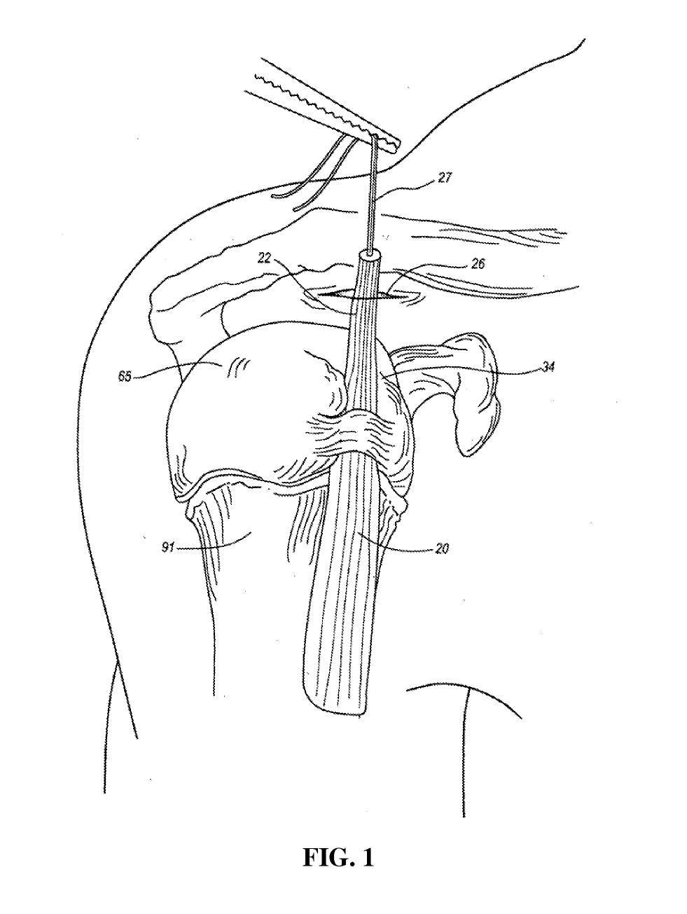

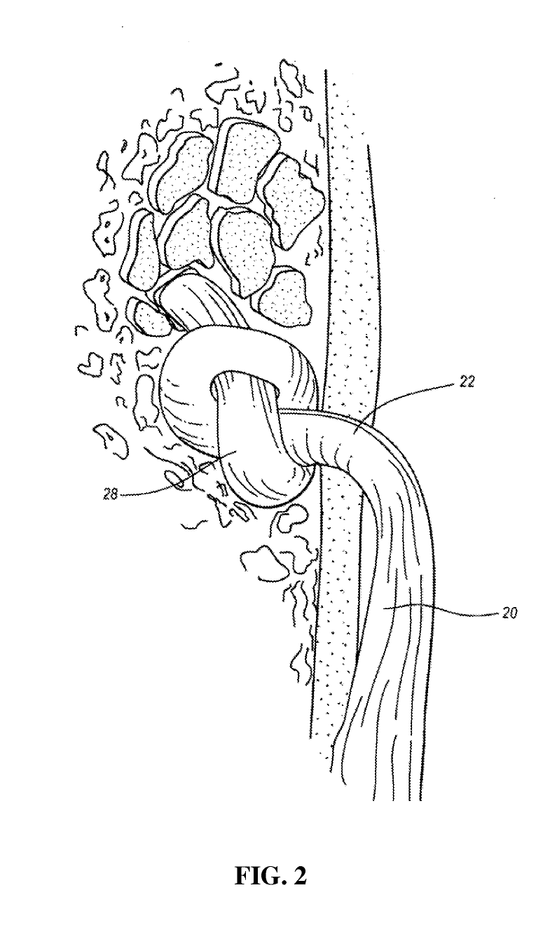

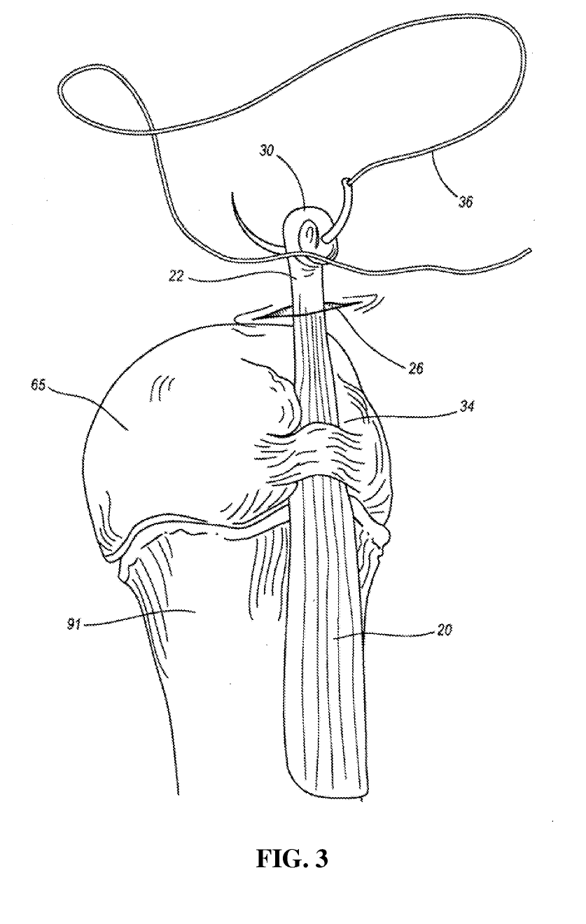

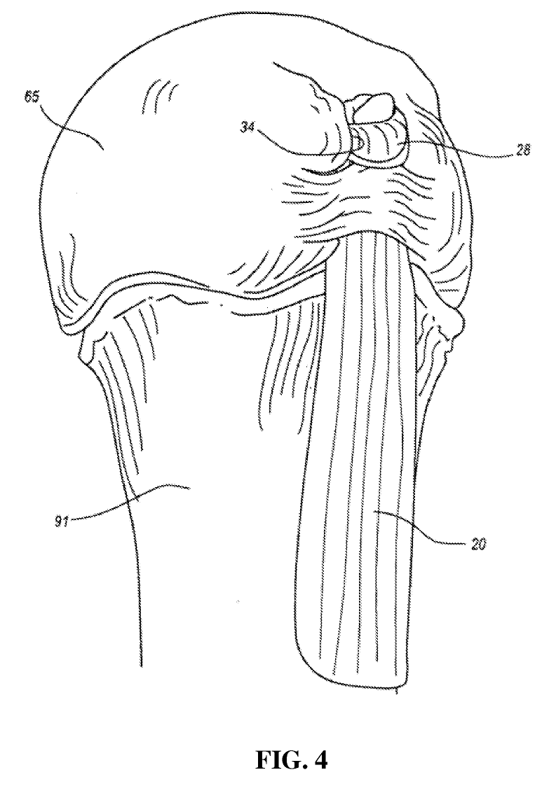

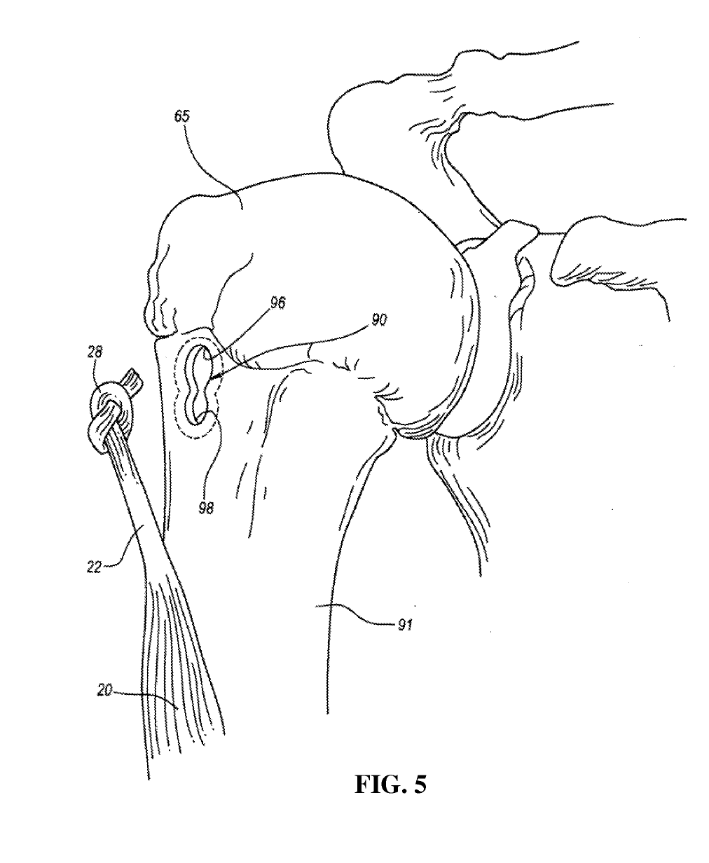

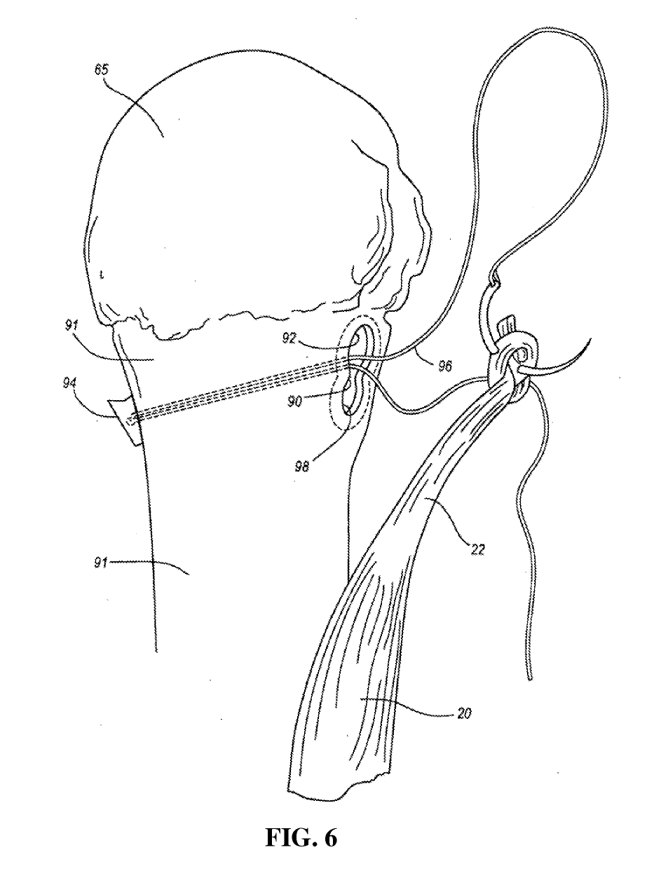

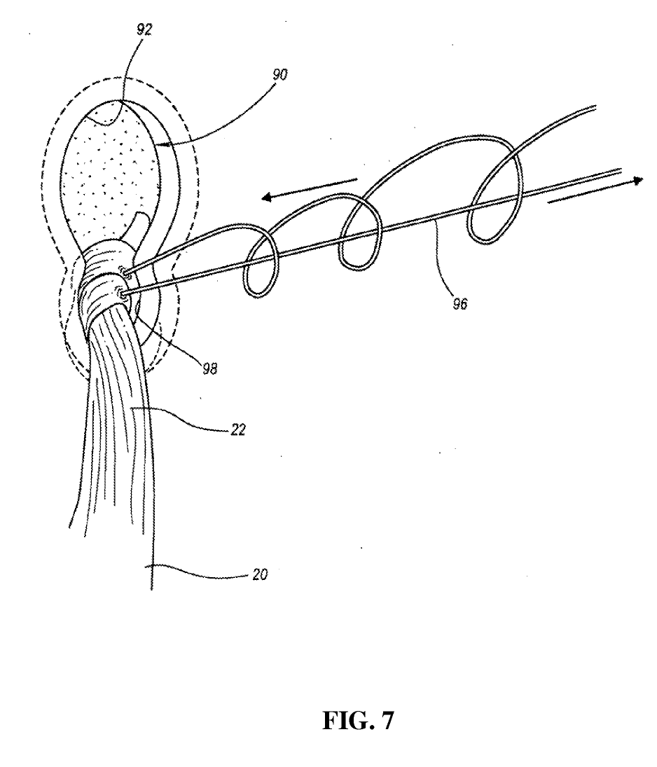

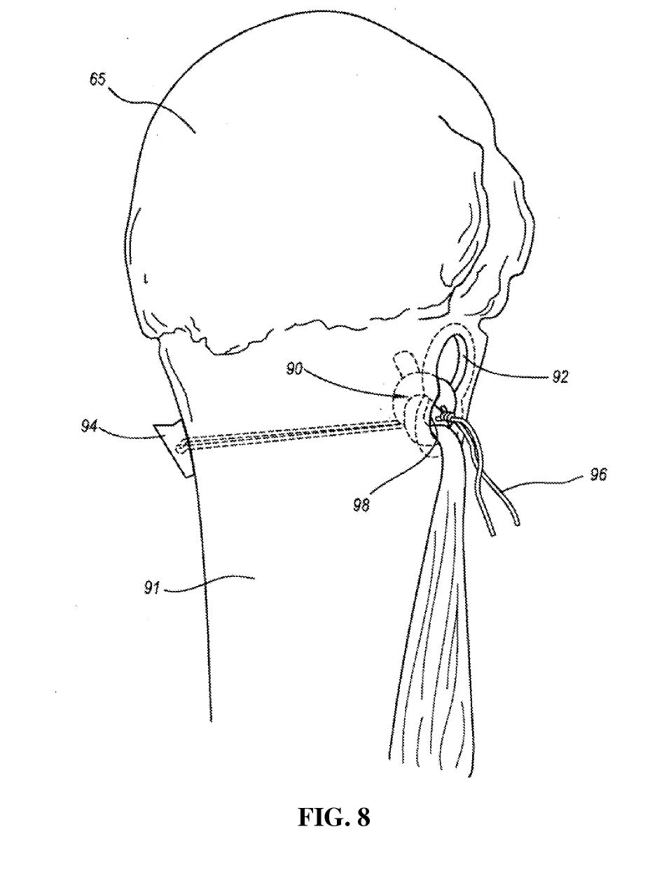

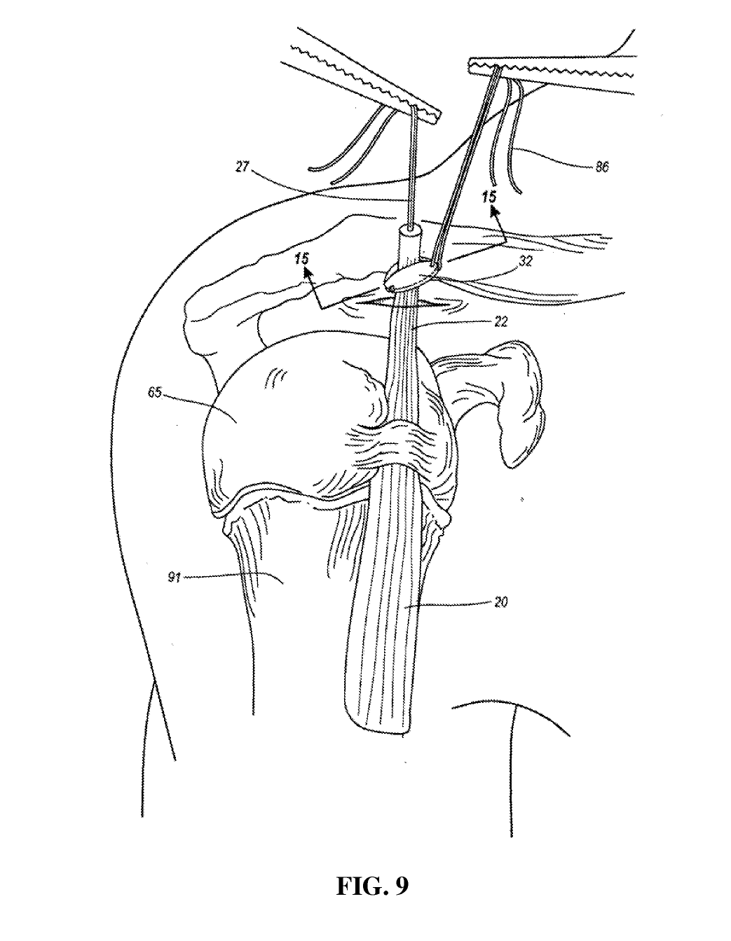

In a preferred embodiment, the process of wedge tenodesis disclosed involves the steps of (1) arthroscopically determining that a repair of a biceps tendon 20 is necessary using conventional arthroscopic procedures, and that a tenodesis is the best procedure for making such repair; (2) arthroscopically separating the head of the biceps tendon from the superior labium; (3) (as one embodiment shown in FIG. 1) pulling the separated end 22 of the biceps tendon 20 through a portal 26 with a suture 27 and trimming the end 22 of the tendon 20; (4) selecting a wedge to be used from a tied knot wedge 28, FIG. 2, a sutured knot wedge 30, FIG. 3, or a button clamp wedge 32, FIG. 9 (and other example embodiments as shown in FIGS. 9-21), to be affixed to the separated end 22 of the tendon 20; (5) (as one embodiment shown in FIG. 9) affixing the selected wedge to the separated end 22 of the tendon 20; and (6) positioning the selected wedge in the bicipital groove 34 (shown in FIG. 3) of the patient while allowing the biceps tendon 20 to retract within the bicipital groove 34 until held by engagement of the wedge with the top of the bicipital groove.

Selection of the wedge used from the tied knot wedge 28, the sutured knot wedge 30, or a button clamp wedge 32 will be based on recognized criteria and the experience of the surgeon. A tied knot wedge 28, if used, is affixed to the separated end 22 of the biceps tendon 20 by forming an overhand knot in the separated end of the biceps tendon. A tied knot will preferably be selected for use if the detached end of the biceps tendon is not severely frayed, torn or otherwise damaged. The sutured knot wedge 30 may be selected and used if the detached end 22 of the biceps tendon 20 is somewhat torn and frayed, but is in good enough condition to be rolled and secured in the rolled state by one or more sutures 36 placed through the rolled end.

The button clamp wedge 32 is selected when the surgeon performing the operation may lack sufficient experience with knot tying or suturing of a rolled end of the biceps tendon. The button clamp wedge is suitable for use as a replacement for either of the identified knot wedges, but is generally selected when the biceps end 22 is in good condition.

The button clamp wedge 32 is easier to install and has proven fully as effective in biceps tenodesis surgery as have either of the identified knot wedges. Consequently, the button clamp wedge may be preferred by even experienced surgeons, whenever a wedge tenodesis is performed and such a clamping button clamp wedge is available for use.

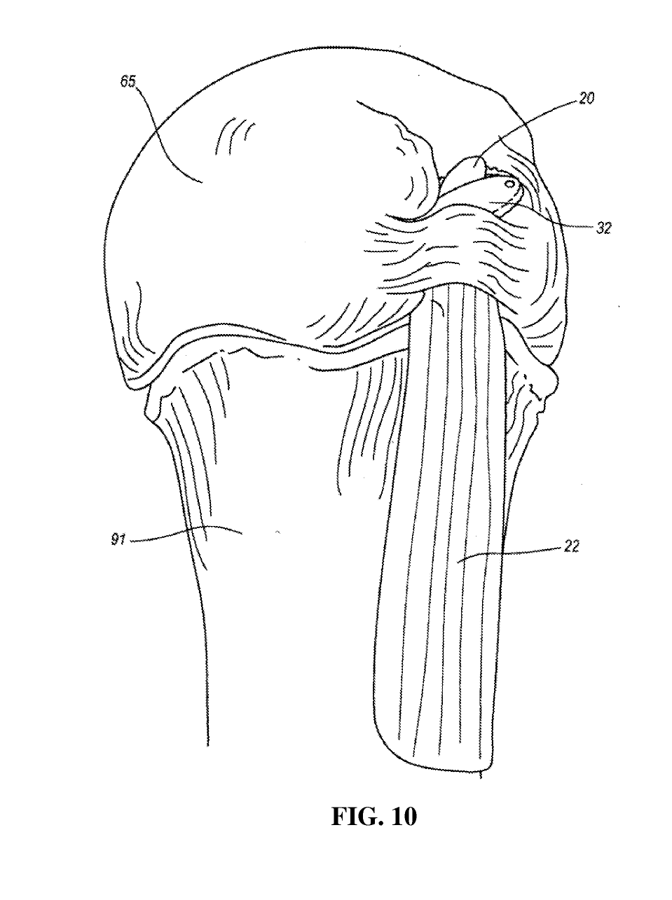

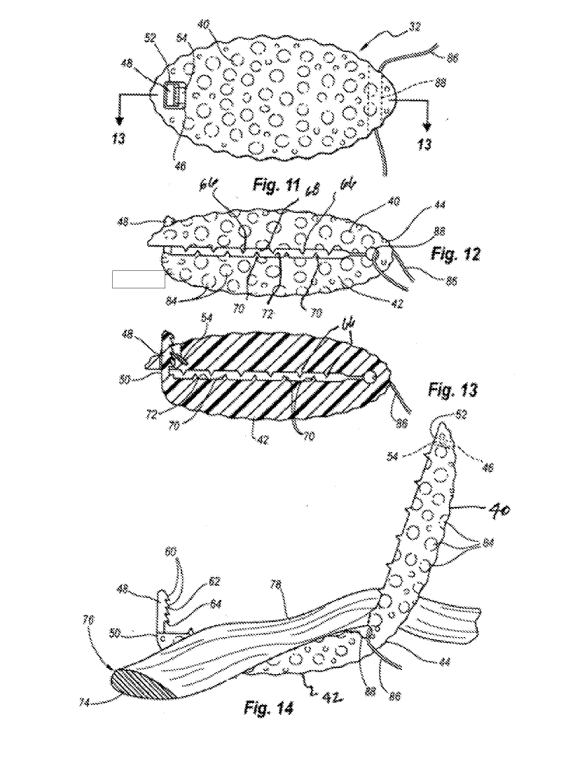

Embodiments of a button clamp wedge or portions thereof, are shown at least in FIGS. 11-21. As shown in FIGS. 11-15, the wedge can be formed with a top portion 40 and a bottom portion 42 interconnected by a hinge 44 proximal to the interconnecting ends of the top and bottom portion. The end of top portion 40 opposite the hinge 44 and the top portion interconnection end has a hole 46 formed therethrough. A rack 48 is formed to extend from the end of bottom portion 42 and is connected to the bottom portion by a hinge 50 that will allow the post to be pivoted towards the top portion 40 and to extend into hole 46 when the top portion 40 is pivoted at hinge 44 to overlie the bottom portion 42. The entrance 52 to hole 46 has a flange 54 extending inwardly thereof. Flange 54 extends angularly into hole 46 from the entrance 52.

Rack 48 has teeth 60 projecting from one side and spaced along the length of the rack 48. Each tooth has an inclined guiding surface 62 extending from one side of the rack upwardly along the length of the rack. An abrupt holding surface 64 extends from the outer edge of each tooth normal to the rack 48.

Spaced apart parallel ribs 66 extend across the inner surface 68 of the top portion 40. Each rib has a triangular cross-sectional configuration with the apex of the rib extending into the space between top and bottom portions when the top portion 40 is folded over the bottom portion 42. Similar spaced apart ribs 70 are formed to extend across the inner surface 72 of bottom portion 42. The ribs 66 and 70 are spaced such that the ribs 66 on the top portion extend between and are spaced to be staggered with respect to the ribs 70 on the bottom portion when the top portion is pivoted to overlie the bottom portion.

In use, the clamping button clamp wedge 32 is affixed to the separated end 22 of a biceps tendon, by positioning the bottom portion 42 of the clamp at one side 74 of the tendon 20 and pivoting the top portion 40 to extend over an opposite side 78 of the biceps tendon.

A locking rack 48 pivots from the bottom portion 42 and as the top and bottom portions are clamped together, with the biceps tendon between them, the rack extends into hole 46. Continued compression of the top and bottom portions 40 and 42 forces the free end of the locking rack 48 further into hole 46, with the engagement of the inclined surfaces 62 of the teeth 60 allowing insertion of the rack past the angled flange 54 extending into the hole 46. Engagement of abrupt surfaces 64 of the teeth with the flange 54 prevents withdrawal of the rack 48 from hole 46. The biceps tendon 20 is thus clamped securely between the top and bottom portions 40 and 42. The apex of the ribs 66 and 70 of the top and bottom portions 40 and 42 are pressed into the biceps tendon and the clamp is secured to the tendon.

In most instances it will be advantageous to have the clamping button clamp wedge 32 extending diagonally across the clamped biceps tendon since this will allow the button to clamp to a significant portion of undamaged tendon and will reduce the possibility that the button clamp wedge will cut or damage an already weakened section of the biceps tendon being repaired.

The illustrated button clamp wedge 32 with the top and bottom portions 40 and 42 clamped together has a general appearance of a football having all outer surfaces “roughened” as shown at 84 to provide a rough surface for better holding action when the button is wedged into the bicipital groove 34.

When the button clamp wedge is used, the long head of the biceps tendon being separated is excised from the superior labrum, and is pulled through a cannula and a portal in the manner previously described in connection with the previously described knot wedge tenodesis.

A suture 86 is passed through the suture hole 88 provided at the hinge 44 between the top and bottom portions 40 and 42. The button clamp wedge 32 is clamped on the end 22 of the biceps tendon and the tendon, with button clamp wedge thereon is released to be guided into wedging position in the bicipital groove 34 at the head 65 of the humerus. If deemed necessary, the bicipital groove may be roughened prior to placement of the button clamp wedge in the groove. In some embodiments, the suture 86 can be used to guide the button clamp wedge into position.

Embodiments of clamshell type button wedge clamps 32, as shown in FIGS. 11-15 are preferably molded in one piece from a suitable plastic material, such as but not limited to PLA plastic.

The wedge tenodesis provides for biceps tendon repair using known arthroscopic technology and avoidance of the incisions necessary for biceps tenotomy. Consequently, wedge tenodesis can be performed faster and without the same degree of surgical experience necessary to the repair of biceps tendons using other established repair procedures.

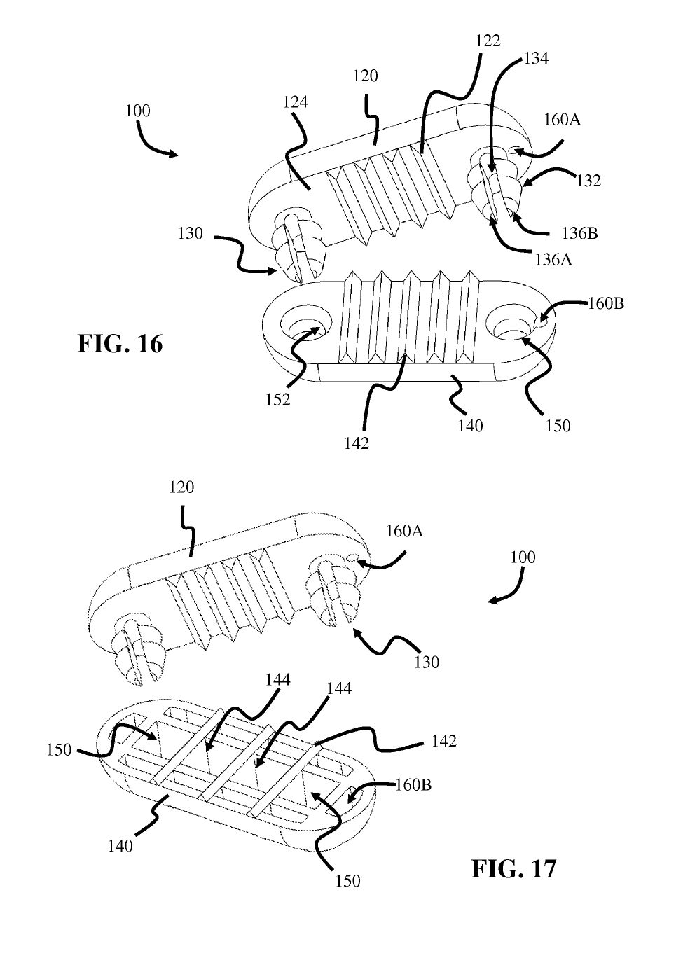

An alternative embodiment of the button comprises a button clamp wedge with multiple pieces or multiple portions. For example, and not for limitation, FIG. 16 illustrates one embodiment of a button 100 where the first top portion 120 has multiple protrusions 130 extending from the inner surface 124 of the first portion. In this example, and not for limitation, the protrusions shown are cylindrical posts extending generally perpendicular to the inner surface 124 of the button first top portion. The posts have a means to lock or retain the posts within cooperating recesses in the second portion. The means to lock shown comprises a forked post formed from multiple posts sections, 136A and 136B, having one or more ridges or teeth 132 extending from the outer surface of the posts. The multiple parallel posts are flexible and separated by a gap 134. The gap 134 and flexibility allow the posts to flex at their distal ends into the gap 134, reducing the outer profile of the post 130 at the distal end. The post sections 136A and 136B also have some rigidity to allow the post sections to be biased into a position where the outer profile of the post 130 can be expanded allowing the teeth 132 to engage mating teeth in the second portion recesses. The second bottom portion 140 has one or more recesses 150 shaped to receive the protrusions 130 of the first portion. In the embodiment shown, the recess is a generally cylindrical recess. Within the recesses are mating teeth 152 extending into the recess 150 that mate to retain and lock the post teeth 132 on the first portion protrusions. When the protrusions 130 of the first portion 120 are inserted into the recesses 150 of the second portion, the post sections 136A and 136B flex into the gap 134, reducing the profile of the post 130 allowing the post teeth 132 to pass the recess teeth 152. Once the apex of a post teeth 132 passes an apex of a recess teeth 152, the bias of the post sections 136A and 136B expands the profile of the post 130 securing the post in the recess 150 and therefore securing the first top portion 120 to the second bottom portion 140. As shown, multiple teeth on the post or in the recess can provide multiple points of securing the portions allowing for multiple tissue sizes to be accommodated by the button. It is understood that although locking means described includes mating teeth and flexible post sections, other means of locking the posts are contemplated such as, but not limited to: rigid or flexible teeth mating with expansion rings, snap rings, or other teeth; mating flanges on both portions; adhesives; or frictional attachment such as can be obtained by luer tapers.

As shown in FIG. 16, similar to the other embodiment described herein, both portions have gripping means to grip the tissue and are externally shaped to help the button lodge in the bicipital groove. The gripping means in this embodiment comprise ridges 122 and 142 on the first and second portions that alternate between each other when the portions are secured together. In the embodiment shown, the gripping means run perpendicular to the button clamp wedge length defined by a distance between the portion ends. As shown, the gripping means runs parallel to a measurement of the body width of the button. Although not required, this example alignment of the gripping means can provide a gripping means that holds the tendon with less stress on the tissue because the ribs of the button generally run in parallel with the fibers of the tendon. This button, when clamped on a tendon, would function similar to the embodiments described above and shown in FIGS. 11-15.

As described above, some embodiments also have a rough outer surface and a through hole (160A and 160B) to receive a traction suture. When used with a through hole, the traction suture can easily be removed by having a cut somewhere in the suture and pulling one end of the suture.

FIG. 17 illustrates another embodiment of a button clamp wedge 100 where the second bottom portion 140 comprises a multiple partitioned portion. The recesses/partitions 150 are able to receive and lock the protrusions 130 of the first portion. The second portion has gripping means to grip the tissue such as ridges 142. Additionally, the additional openings, such as 144 shown, in the second portion created by the partitions also provide additional frictional surfaces to assist the gripping means to grip the tissue. This embodiment may include through holes (160A and 160B) for guide tools such as a traction suture.

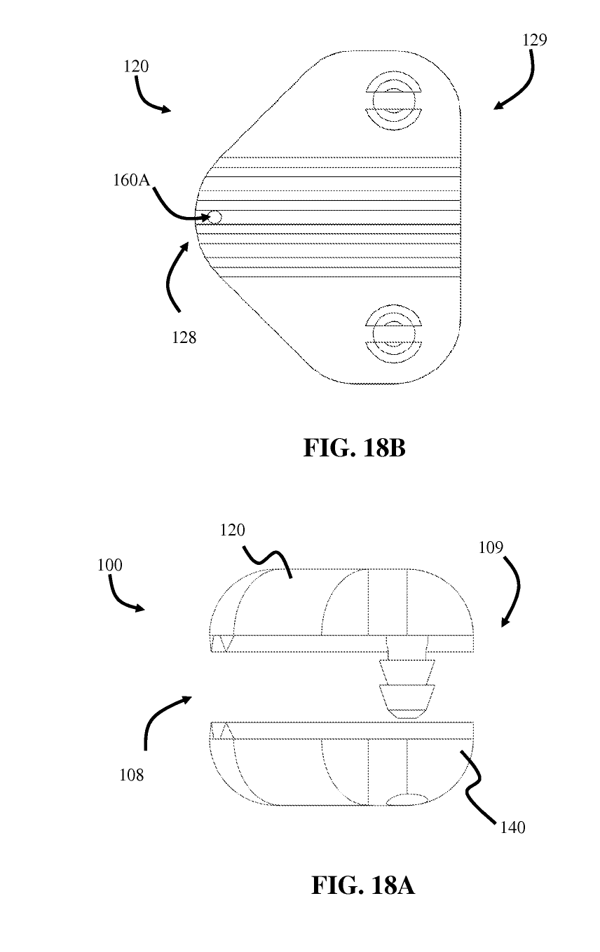

FIGS. 18A and 18B illustrate another embodiment of the button clamp. In this embodiment, the shape of the button clamp wedge is triangularly wedge shaped to help securely lodge the button clamp wedge in the bicipital groove. FIG. 18A illustrates a side profile while FIG. 18B shows a bottom view of the first portion of the button clamp wedge showing the wedge shape with the front side 129 of the portion being more narrow than the trailing side 129.

Referring to FIG. 18A, with this shape, the more pointed end of the wedge shape (indicated at 108 in FIG. 18A) of the button clamp wedge 100 can be inserted into the bicipital groove initially with the larger profile end of the wedge shape 109 trailing to provide a wedge to lodge the button clamp wedge 100 into the groove. As shown in this embodiment, but as can apply with other embodiments, the through hole for the traction suture can be through both the first and second body portions. This allows the suture to keep the components together during surgery, to act as both a guide for the positioning of the button clamp wedge as well as to help secure the two portions together. For example, during the procedure, the suture keeps the two portions in close proximity to each other and when the procedure is complete, the legs of the traction suture can be tied together to provide an additional means to secure the two portions together. Similar to the other embodiments, gripping means and locking means are provided.

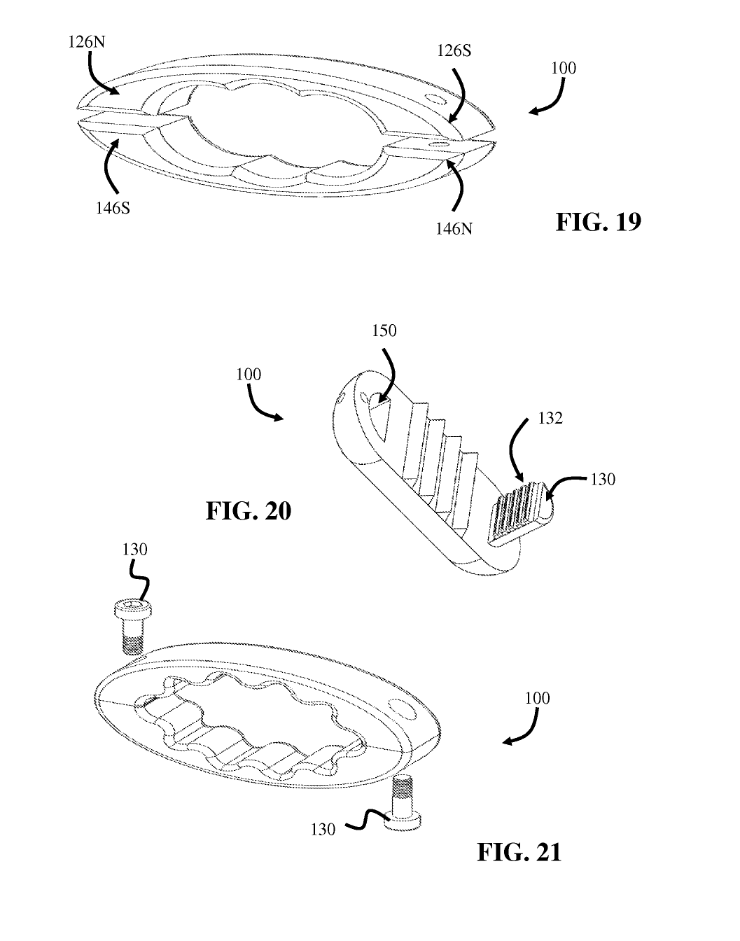

FIG. 19 illustrates an embodiment where the means for locking the two portions together is a magnetic field on both ends of both portions. As shown, the first portion is curved with one end having a magnetic pole of “North”, 126N and the other end of the portion being a “South” pole, 126S. In this embodiment, the second portion is similarly magnetized with a North pole of 146N and South pole of 146S and when the two portions are placed with the opposite charged ends adjacent to each other the portions attract and connect. It is understood that embodiments can comprise two portions attracted magnetically while only one of the portions may be magnetized. For example, one portion may be magnetized but the other portion may have a metallic portion that is attracted to the magnetized portion. Similarly, only one section of a portion may have a magnetized section while the other portion has a metallic section. The magnetized sections are provided to have suitable shape, size, polarity, and magnetic intensity to provide a means to retain or otherwise lock the two portions together. In general, the magnetic sections of the portions according to embodiments of the invention can be made of permanent magnets. Examples of such permanent magnets preferably include, but not limited to, rare earth cobalt magnets (e.g., samarium-cobalt, SmCo), neodymium, ceramic, alnico and rare earth iron boron magnets (e.g., sintered neodymium-iron-boron, NdFeB). Magnets according to the invention may further include diamagnetic, paramagnetic, ferromagnetic, anti-ferromagnetic, and/or ferrimagnetic material, and/or any other materials that may be incorporated to create the appropriate magnetic field. One example of such magnet sections is a magnetized piece inserted into one of the portions. Steel or other ferromagnetic material may also be used to complete a circuit by contacting the magnets on their back surfaces.

FIG. 20 illustrates an embodiment where there is one portion design that can be used for either the first and second portion. In this design, the portion has both a protrusion 130 and a recess 150. When two of these embodiments are used together, the inner surfaces are placed adjacent to each other with one portion's protrusion opposite the other portion's recess and the two portions can be secured to each other to retain the tissue. In this embodiment, the protrusion has flexible flanges 132, such as teeth, and the recess is a recess having mating teeth to hold the flanges 132 and lock the portions together.

FIG. 21 illustrates another embodiment of a button clamp wedge 100 having two generally curved shaped portions having protrusions that comprise threaded screws 130 that secure and lock the first portion to the second portion. Although the embodiment shown as one screw going through one portion to be received in a threaded recess in the other portion, it is understood that embodiments may have both screws starting in one portion and being received by the other. In these embodiments, the screws have heads that are too large to pass through a recess in the first portion such that tightening the screws secures the first portion to the second portion.

For embodiments such as FIGS. 19-21, through holes for traction sutures can be placed on both ends of these portions and the embodiment can benefit from the fact that the portions generally minor each other. This design allows one portion design to perform the function of both the first and second portion simplifying the use and manufacturing of the button clamp wedge.

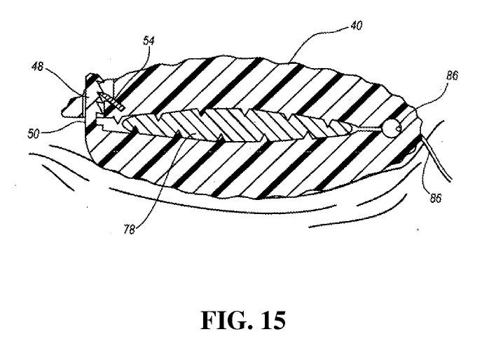

Embodiments of the button wedge clamp 32, as shown in FIGS. 15-21 can be made from any suitable surgical material such as but not limited to PLA plastic, titanium or stainless steel.

It is understood that although the above descriptions include discussions and illustrations of protrusions and recesses, it is understood that other means for locking the two portions together are contemplated. For example, the protrusions can be comprise, but are not limited to, screws, clips, flanges, hooks or any extension that can mate with and connect one portion with another portion. For example and not for limitation, the recess can be comprised of a threaded recess, through-hole with ridges or teeth, an indentation or any means to connect with the protrusion and hold the portions together.

It is also clear that if a knot or button clamp wedge shall fail to wedge tightly in the bicipital groove, the wedge used, whether knot or button clamp wedge, can thereafter be alternatively fitted into and be secured in a keyhole slot 90 formed in the humeras 91. Should this become necessary, the keyhole slot 90 is formed in known fashion and with a bore hole extending through the humeras in known fashion from the keyhole slot 90. The wedge, whether knot or button clamp wedge, can also have the suture line 96 passed therethrough. The knot or button clamp wedge is pulled into the large opening 92 of the keyhole, in known fashion, using an anchor 94 and suture 96 and is allowed to drop into the smaller opening 98 of the keyhole. The wedge is then held in the keyhole to secure the large head of the tendon.

For embodiments that utilize a button wedge clamp, the traction suture 86 that is received in the clamp can be used to guide and pull the button clamp wedge and tendon into the large opening of the keyhole.

Although preferred forms of our invention have been herein disclosed, it is to be understood that the present disclosure is by way of example and that variations are possible without departing from the subject matter coming within the scope of the following claims, which subject matter we regard as our invention.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention. Although this invention has been described in the above forms with a certain degree of particularity, it is understood that the present disclosure has been made only by way of example and numerous changes in the details of construction and combination and arrangement of parts may be resorted to without departing from the spirit and scope of the invention.

We claim:

1. A button clamp wedge for use in performing wedge tenodesis of a biceps tendon comprising a generally football shaped body comprising a first top portion and a second bottom portion;
a hinge interconnecting an interconnecting end of the top portion to an interconnecting end of the bottom portion;
means for locking a locking end of the top portion to a locking end of the bottom portion;
the body having a length defined by a distance between the interconnecting and locking ends and a body width;
gripping means extending from the top portion, each of the gripping means having an apex forming at least one rib across the body width extending towards the bottom portion when the portions are locked together; and
gripping means extending from the bottom portion, each of the gripping means having an apex forming at least one rib across the body width extending towards the top portion, when the portions are locked together the gripping means on the top portion and the gripping means on the bottom portion alternating along the length of the body.

2. A button clamp wedge as in claim 1, wherein the means for locking the locking end of the top portion to the locking end of the bottom portion, comprises:
a rack having a first end pivotally connected to the locking end of the bottom portion;
a receiving opening formed at the locking end of the top portion; and
means to secure a second end of the rack in the receiving opening.

3. A button clamp wedge as in claim 2, wherein the means to secure the second end of the rack within the receiving opening comprises:
a flange extending angularly into the receiving opening; and
a plurality of spaced apart sloped teeth extending from the rack, each of the teeth extending from the rack including an abrupt surface to engage the flange extending into the receiving opening to prevent withdrawal of the rack from the receiving opening.

4. A button clamp wedge as in claim 3, wherein:
the teeth on the rack and the flange extending into the receiving opening have sufficient flexibility to allow insertion of the second end of the rack and the teeth thereon into the receiving opening, and are sufficiently rigid to prevent withdrawal of the rack from the receiving opening.

5. A button clamp wedge as in claim 1, wherein:
the football shaped body is sized to replace a rolled end of a biceps tendon; and
the gripping means is adapted to clamp on the biceps tendon in an arthroscopic wedge tenodesis of the biceps tendon.

6. A button clamp wedge as in claim 1 wherein an outer surface of the top portion and an outer surface of the bottom portion is a rough surface.

7. A button clamp wedge as in claim 1 wherein the apex of the gripping means comprises an apex of a rib having a substantially triangular cross-sectional configuration extending from an interior surface.

8. A button clamp wedge as in claim 1 further comprising a hole at one of the interconnecting and locking ends of the top portion and the bottom portion.

9. A button clamp wedge as in claim 8 further comprising a traction suture and wherein the hole is defined at the interconnecting ends of the top portion and the bottom portion and capable of receiving the traction suture and capable of allowing removal of the traction suture when the portions of the clamp are locked together.

10. A button clamp wedge for use in performing wedge tenodesis of a biceps tendon comprising:
a clamp body having a separable first and second portion;
the first and second portions each having an exterior and an interior surface;
a means for locking the first and second portions together;
a gripping means extending from the interior surface of the first and second portion; and
each of the gripping means having an apex extending towards the other portion when the portions are locked together.

11. A button clamp wedge as in claim 10, wherein when the portions are locked together the apex of the gripping means on the first portion and the apex of the gripping means on the second portion alternate along a length of the portions.

12. A button clamp wedge as in claim 10, wherein the exterior surface of the portions is roughened.

13. A button clamp wedge as in claim 10 further comprising a suture hole in at least one of the portions.

14. A button clamp wedge as in claim 10 wherein the means for locking the first and second portions together comprises a magnetized north and south pole section on each of the portions whereby placing oppositely magnetized sections adjacent to each other cause the sections to attract each other and lock the first and second portion together.

15. A button clamp wedge as in claim 10 wherein the means for locking the first and second portions together comprises:
at least one magnetized section on one of the portions; and
at least one metallic section on the other portion whereby placing the at least one magnetized section adjacent to the at least one metallic section causes the magnetized section to attract to the metallic section and lock the first and second portions together.

16. A button clamp wedge as in claim 10, wherein the means for locking the first and second portions together, comprises:
a protrusion extending away from the body of the first portion;
a recess extending into the body of the second portion; and
the protrusion extending from the first portion having a shape to be received and frictionally held in the recess extending into the second portion whereby the first and second portions can be connected.

17. A button clamp wedge as in claim 16 wherein:
the protrusion comprises a cylindrical post extending from the interior surface of the first portion;
the recess comprises a cylindrical recess extending into the interior surface of the second portion; and
the recess being malleable and having a size slightly larger than the protrusion whereby the recess is capable of frictionally engaging the protrusion and frictionally connecting the first and second portions.

18. A button clamp wedge as in claim 16, wherein:
the protrusion comprises a forked post extending from the interior surface of the first portion;
the recess comprises a cylindrical recess extending into the interior surface of the second portion; and
the forked post having flanges whereby each recess is capable of frictionally engaging the flanges and frictionally connecting the first and second portions.

19. A button clamp wedge as in claim 16 wherein:
the protrusion having at least one tooth extending from the protrusion and each of the tooth including an abrupt surface;
the recess comprises a receiving opening extending into the interior surface of the second portion; and
the recess having at least one tooth extending into the receiving opening whereby the tooth can engage the tooth of the receiving opening to prevent withdrawal of the protrusion from the receiving opening.

20. A method for arthroscopic biceps tenodesis comprising the steps of:
arthroscopically separating a long head of a biceps tendon from attachment to a bone;
placing a button clamp wedge at a proximal end of the long head of a biceps tendon;
allowing the tendon to slide through a glenohumeral joint; and
lodging the button clamp wedge in a bicipital groove before the tendon slides fully through the glenohumeral joint.

21. A method as in claim 20, wherein the button clamp wedge comprises:
a first and second portion;
the first and second portions are separable and capable of being connected to each other; and
the step of placing the button clamp wedge comprises placing the biceps tendon between the first and second portions of the button clamp wedge and connecting the portions.

22. A method as in claim 20, wherein the first and second portions have a roughened exterior surface enhancing the step of lodging the button clamp wedge in the bicipital groove.
